SEQUENCE LISTING (9 sequences)

SEQ ID NO: 1 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 2 - protein, 412 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYDFEKVGYYNPTDGTWTIKVVSYSGSANYQVDVVSDGSLSQPGSS

SEQ ID NO: 3 - protein, 425 aa, Thermococcuc sp PK
MEFNKVFSLLLVFVVLGATAGIVGAAPAEKARVIITIDKDFNENSVFALGGNVVARGKVFPIVIAELPPRAIERLKNAKGVVRVEYDAEAHILKGKPPGTGKPKPSQPAQTIPWGIERIKAPDAWSITDGSSGGVIEVAILDTGIDYDHPDLAANLAWGVSVLRGKVSTNPKDYKDQNGHGTHVAGTVAALNNDIGVVGVASAVEIYAVRVLDASGRGSYSDIILGIEQALLGPDGVLDSDNDGVIVGDPDDDAAEVISMSLGGSSDVQAFHDAIIEAYNYGVVIVAASGNDGASSPSYPAAYPEVIAVGATDSDDQVPWWSNRGVEVSAPGVDILSTYPDDTYETLSGTSMATPHVSGVVALIQAAYYNKYGYVLPVGTFGDLTTSTVRGILHVTADDLGSSGWDADYGYGIVRADLAVQAAIS

SEQ ID NO: 4 - protein, 177 aa, Thermoascus aurantiacus
TRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 5 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 6 - protein, 591 aa, Talaromyces emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 7 - protein, 556 aa, Gloeophyllum sepiarium
QSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 8 - protein, 559 aa, Gloeophyllum trabeum
QSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 9 - protein, 583 aa, Artificial
  Hybrid alpha-amylase
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

(19) United States
(12) Patent Application Publication
(10) Pub. No.: US 2018/0208916 A1
(43) Pub. Date: Jul. 26, 2018

(54) ALPHA-AMYLASE VARIANTS AND POLYNUCLEOTIDES ENCODING SAME

(71) Applicant: NOVOZYMES A/S, Bagsvaerd (DK)

(72) Inventors: Carsten Andersen, Vaerloese (DK); Jameel Shihadeh, Raleigh, NC (US)

(73) Assignee: NOVOZYMES A/S, Bagsvaerd (DK)

(21) Appl. No.: 15/746,712

(22) PCT Filed: Jul. 20, 2016
(86) PCT No.: PCT/US2016/043074

§ 371 Date: Jan. 22, 2018

Related U.S. Application Data
(60) Provisional application No. 62/196,059, filed on Jul. 23, 2015.

(51) Int. Cl. C12N 9/28; C12P 19/14; C12N 9/54; C12P 19/02; C12N 9/34

(52) U.S. Cl. CPC C12N 9/2417; C12P 19/14; C12Y 302/01001; C12P 19/02; C12N 9/2428; C12N 9/54

(57) ABSTRACT

The present invention relates to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 85%, at least 90%, at least 95%, at least 96%, at least 97%, at least 98%, or at least 99%, but less than 100% sequence identity to the mature polypeptide of SEQ ID NO: 1, and wherein the variant comprises at least one of the following combination of substitutions: V59A+E129V+K177L+R179E+H208Y+K220P+N224L+Q254S; V59A+E129V+K177L+R179E+Q254S+M284V; or V59A+E129V+K177L+R179E+V212T+Q254S+M284V (using SEQ ID NO: 1 for numbering); and wherein the variants have alpha-amylase activity.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to alpha-amylase variants, polynucleotides encoding the variants, methods of producing the variants, and methods of using the variants.

### Description of the Related Art

Alpha-amylases (alpha-1,4-glucan-4-glucanohydrolases, E.C. 3.2.1.1) constitute a group of enzymes, which catalyze hydrolysis of starch and other linear and branched 1,4-glucosidic oligo- and polysaccharides.

Alpha-amylases are used commercially for a variety of purposes such as in the initial stages of starch processing (e.g., liquefaction); in wet milling processes; and in alcohol production from carbohydrate sources. They are also used as cleaning agents or adjuncts in detergent matrices; in the textile industry for starch desizing; in baking applications; in the beverage industry; in oil fields in drilling processes; in recycling processes, e.g., for de-inking paper; and in animal feed.

Fermentation products, such as ethanol, are typically produced by first grinding starch-containing material in a dry-grind or wet-milling process, then degrading the material into fermentable sugars using enzymes and finally converting the sugars directly or indirectly into the desired fermentation product using a fermenting organism. Liquid fermentation products are recovered from the fermented mash (often referred to as “beer mash”), e.g., by distillation, which separate the desired fermentation product from other liquids and/or solids.

An alpha-amylase from Bacillus stearothermophilus is disclosed in WO 99/19467 as SEQ ID NO: 3, and variants thereof have been disclosed in WO 96/23873, and WO 99/19467. Further variants of the Bacillus stearothermophilus alpha-amylase are disclosed in WO 2011/082425.

WO 2012/088303 (Novozymes) discloses processes for producing fermentation products by liquefying starch-containing material at a pH in the range from 4.5-5.0 at a temperature in the range from 80-90° C. using a combination of alpha-amylase having a T½ (min) at pH 4.5, 85° C., 0.12 mM CaCl₂) of at least 10 and a protease having a thermostability value of more than 20% determined as Relative Activity at 80° C./70° C.; followed by saccharification and fermentation.

WO 2013/082486 (Novozymes) discloses processes for producing fermentation products by liquefying starch-containing material at a pH in the range between from above 5.0-7.0 at a temperature above the initial gelatinization temperature using an alpha-amylase variant.

The present invention provides alpha-amylase variants with improved properties compared to its parent.

### SUMMARY OF THE INVENTION

The present invention relates to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 85%, at least 90%, at least 95%, at least 96%, at least 97%, at least 98%, or at least 99%, but less than 100% sequence identity to the mature polypeptide of SEQ ID NO: 1, and wherein the variant comprises at least one of the following combination of substitutions:

### V59A+E129V+K177L+R179E+H208Y+K220P+N224L+Q254S;

### V59A+E129V+K177L+R179E+Q254S+M284V; or

V59A+E129V+K177L+R179E+V212T+Q254S+M284V (using SEQ ID NO: 1 for numbering); and wherein the variants have alpha-amylase activity.

The present invention also relates to polynucleotides encoding the variants; nucleic acid constructs, vectors, and host cells comprising the polynucleotides; and methods of producing the variants.

The present invention further relates to a process for producing fermentation products from starch-containing material comprising the steps of: a) liquefying the starch-containing material at a temperature above the initial gelatinization temperature using an alpha-amylase according to the invention; b) saccharifying using a glucoamylase; c) fermenting using a fermenting organism.

The present invention also relates to a composition comprising the variant alpha-amylase of the invention.

### Definitions

Alpha-amylases (alpha-1,4-glucan-4-glucanohydrolases, E.C. 3.2.1.1) are a group of enzymes, which catalyze the hydrolysis of starch and other linear and branched 1,4-glucosidic oligo- and polysaccharides. For purposes of the present invention, alpha-amylase activity is determined according to the procedure described in the Examples. In one aspect, the variants of the present invention have at least 20%, e.g., at least 40%, at least 50%, at least 60%, at least 70%, at least 80%, at least 90%, at least 95%, or at least 100% of the alpha-amylase activity of the parent alpha-amylase. In one embodiment the parent is the alpha-amylase denoted AA-369.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Fragment: The term “fragment” means a polypeptide having one or more (e.g., several) amino acids absent from the amino and/or carboxyl terminus of a mature polypeptide; wherein the fragment has alpha-amylase activity. In one aspect, a fragment contains around 490 amino acid residues (e.g., amino acids 1-482 to 1-493 of SEQ ID NO: 1). wherein the In one particular embodiment the Bacillus stearothermophilus variant alpha-amylase is truncated, preferably after position 484 of SEQ ID NO: 1, particularly after position 485, particularly after position 486, particularly after position 487, particularly after position 488, particularly after position 489, particularly after position 490, particularly after position 491, particularly after position 492, more particularly after position 493.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. In one embodiment the improved property is reduced viscosity of a liquefied mash when the variants according to the invention are used in liquefaction of ground corn performed at pH 4.8 and 85° C. for 2 hours, compared to using a control (parent) alpha-amylase. At the same time ethanol yield after saccharification and fermentation remained the same as seen for the controls (parent alpha-amylase). In one embodiment the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent alpha-amylase: The term “parent” or “parent alpha-amylase” means any polypeptide with alpha-amylase activity to which an alteration is made to produce the enzyme variants of the present invention.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having alpha-amylase activity.

S8A Protease: The term “58A protease” means an S8 protease belonging to subfamily A. Subtilisins, EC 3.4.21.62, are a subgroup in subfamily S8A, however, the present S8A protease from Thermococcus sp PK is a subtilisin-like protease, which has not yet been included in the IUBMB classification system. The S8A protease according to the invention hydrolyses the substrate Suc-Ala-Ala-Pro-Phe-pNA. The release of p-nitroaniline (pNA) results in an increase of absorbance at 405 nm and is proportional to the enzyme activity. pH optimum=pH 8, and Temperature optimum=60° C.

In one aspect, the S8A protease has at least 20%, e.g., at least 40%, at least 50%, at least 60%, at least 70%, at least 80%, at least 90%, at least 95%, or at least 100% of the protease activity of the mature polypeptide of SEQ ID NO: 2. In one embodiment protease activity can be determined by the kinetic Suc-AAPF-pNA assay as disclosed in example 4.

Variant: The term “variant” means a polypeptide having alpha-amylase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the alpha-amylase activity of the parent alpha-amylase. In one embodiment the parent is the alpha-amylase denoted AA-369.

Wild-type alpha-amylase: The term “wild-type” alpha-amylasemeans an alpha-amylase expressed by a naturally occurring microorganism, such as a bacterium, yeast, or filamentous fungus found in nature.

### Conventions for Designation of Variants

For purposes of the present invention, the mature polypeptide disclosed in SEQ ID NO: 1 is used to determine the corresponding amino acid residue in another alpha-amylase. The amino acid sequence of another alpha-amylase is aligned with the mature polypeptide disclosed as SEQ ID NO: 1, and based on the alignment, the amino acid position number corresponding to any amino acid residue in the mature polypeptide disclosed as SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another alpha-amylase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other alpha-amylase has diverged from the polypeptide of SEQ ID NO: 1 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions.

For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions.

For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions.

For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Multiple Alterations.

Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different Alterations.

Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

### “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to variants of a Bacillus stearothermophilus alpha-amylase disclosed in WO 99/19467 as SEQ ID NO: 3 (SEQ ID NO: 1 herein) having a double deletion of two amino acids in the region from position 180 to 182, e.g., at positions G180, I181 and/or G182, in particular deletion of I181+G182, G180+I181, or G180+G182, preferably I181+G182, and further comprising one of the combinations of substitutions selected from the list:

V59A+E129V+K177L+R179E+H208Y+K220P+N224L+Q254S;

V59A+E129V+K177L+R179E+Q254S+M284V; or

V59A+E129V+K177L+R179E+V212T+Q254S+M284V (using SEQ ID NO: 1 for numbering); and wherein the variants have alpha-amylase activity.

In one embodiment the variants further comprise a substitution of N193F.

The variants according to the invention result in a reduced viscosity of a liquefied mash when using the variants for liquefaction of ground corn performed at pH 4.8 and 85° C. for 2 hours, compared to the control (parent) alpha-amylase. At the same time ethanol yield after saccharification and fermentation remained the same as seen for the controls (parent alpha-amylase). In one embodiment the reduction in viscosity is at least 5%, at least at least 6%, at least 7%, at least 8%, at least 9%, or at least 10%, at least 11%, at least 12%, at least 13%, at least 14%, at least 15%

The control (or parent) alpha-amylase is an alpha-amylase variant having the most substitutions and deletions in common with the variants according to the invention. The control alpha-amylases have been disclosed in WO 2011/082425 and WO 2013/082486.

### Specific Embodiments of Variants

In one embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 85% but less than 100% sequence identity to the polypeptide of SEQ ID NO: 1, and wherein the variant comprises the substitutions: V59A+E129V+K177L+R179E+H208Y+K220P+N224L+Q254S; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 90% but less than 100% sequence identity to the polypeptide of SEQ ID NO: 1, and wherein the variant comprises the substitutions: V59A+E129V+K177L+R179E+H208Y+K220P+N224L+Q254S; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 95% but less than 100% sequence identity to the polypeptide of SEQ ID NO: 1, and wherein the variant comprises the substitutions: V59A+E129V+K177L+R179E+H208Y+K220P+N224L+Q254S; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 96% but less than 100% sequence identity to the polypeptide of SEQ ID NO: 1, and wherein the variant comprises the substitutions: V59A+E129V+K177L+R179E+H208Y+K220P+N224L+Q254S; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 97% but less than 100% sequence identity to the polypeptide of SEQ ID NO: 1, and wherein the variant comprises the substitutions: V59A+E129V+K177L+R179E+H208Y+K220P+N224L+Q254S; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 98% but less than 100% sequence identity to the polypeptide of SEQ ID NO: 1, and wherein the variant comprises the substitutions: V59A+E129V+K177L+R179E+H208Y+K220P+N224L+Q254S; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 99% but less than 100% sequence identity to the polypeptide of SEQ ID NO: 1, and wherein the variant comprises the substitutions: V59A+E129V+K177L+R179E+H208Y+K220P+N224L+Q254S; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In one embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 85% but less than 100% sequence identity to the polypeptide of SEQ ID NO: 1, and wherein the variant comprises the substitutions: V59A+E129V+K177L+R179E+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 90% but less than 100% sequence identity to the polypeptide of SEQ ID NO: 1, and wherein the variant comprises the substitutions: V59A+E129V+K177L+R179E+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 95% but less than 100% sequence identity to the polypeptide of SEQ ID NO: 1, and wherein the variant comprises the substitutions: V59A+E129V+K177L+R179E+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 96% but less than 100% sequence identity to the polypeptide of SEQ ID NO: 1, and wherein the variant comprises the substitutions: V59A+E129V+K177L+R179E+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 97% but less than 100% sequence identity to the polypeptide of SEQ ID NO: 1, and wherein the variant comprises the substitutions: V59A+E129V+K177L+R179E+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 98% but less than 100% sequence identity to the polypeptide of SEQ ID NO: 1, and wherein the variant comprises the substitutions: V59A+E129V+K177L+R179E+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 99% but less than 100% sequence identity to the polypeptide of SEQ ID NO: 1, and wherein the variant comprises the substitutions: V59A+E129V+K177L+R179E+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In one embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 85% but less than 100% sequence identity to the polypeptide of SEQ ID NO: 1, and wherein the variant comprises the substitutions: V59A+E129V+K177L+R179E+V212T+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 90% but less than 100% sequence identity to the polypeptide of SEQ ID NO: 1, and wherein the variant comprises the substitutions: V59A+E129V+K177L+R179E+V212T+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 95% but less than 100% sequence identity to the polypeptide of SEQ ID NO: 1, and wherein the variant comprises the substitutions: V59A+E129V+K177L+R179E+V212T+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 96% but less than 100% sequence identity to the polypeptide of SEQ ID NO: 1, and wherein the variant comprises the substitutions: V59A+E129V+K177L+R179E+V212T+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 97% but less than 100% sequence identity to the polypeptide of SEQ ID NO: 1, and wherein the variant comprises the substitutions: V59A+E129V+K177L+R179E+V212T+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 98% but less than 100% sequence identity to the polypeptide of SEQ ID NO: 1, and wherein the variant comprises the substitutions: V59A+E129V+K177L+R179E+V212T+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In another embodiment the variants according to the invention relate to an alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 99% but less than 100% sequence identity to the polypeptide of SEQ ID NO: 1, and wherein the variant comprises the substitutions: V59A+E129V+K177L+R179E+V212T+Q254S+M284V; and wherein the variant further comprises a double deletion in the region from position 179 to 182, particularly deletion of I181+G182; and wherein the reduction in viscosity compared to the parent alpha-amylase is at least 5%, at least at least 6%, at least 7%, at least 8%, at least 9%, or at least 10%, at least 11%, at least 12%, at least 13%, at least 14%, at least 15%.

In particular the amino acid at position 89 in the variants according to the invention is not R, particularly the amino acid at position 89 is selected from N, D, E, H, S, T, A, or Q, more particularly the amino acid at position 89 is Q.

The variants according to the present invention may in one embodiment further comprise the substitution N193F.

In a still further embodiments the variant alpha-amylase may be truncated at the C-terminal, preferably to have around 490 amino acids, such as from 482-493 amino acids. In one embodiment the Bacillus stearothermophilus variant alpha-amylase is truncated, preferably after position 484 of SEQ ID NO: 1, particularly after position 485, particularly after position 486, particularly after position 487, particularly after position 488, particularly after position 489, particularly after position 490, particularly after position 491, particularly after position 492, more particularly after position 493.

The variants may further comprise one or more additional alterations at one or more (e.g., several) other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for alpha-amylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The polypeptide may be a hybrid polypeptide in which a region of one polypeptide is fused at the N-terminus or the C-terminus of a region of another polypeptide.

The parent may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Polynucleotides

The present invention also relates to polynucleotides encoding a variant of the present invention.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436). However, any method known in the art for introducing DNA into a host cell can be used.

### Methods of Production

The present invention also relates to methods of producing a variant, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Compositions

The present invention also relates to compositions comprising a variant alpha-amylase of the present invention.

The compositions may comprise a variant alpha-amylase of the present invention as the major enzymatic component, e.g., a mono-component composition. Alternatively, the compositions may comprise multiple enzymatic activities, such as one or more (e.g., several) enzymes selected from the group consisting of protease, glucoamylase, beta-amylase, pullulanase. In a particular embodiment the composition comprises a variant alpha-amylase of the present invention and a protease, particularly a protease from Pyrococcus sp., or Thermococcus sp., or a protease from Thermoascus aurantiacus.

In one embodiment the protease is selected from a protease S from Pyrococcus furiosus shown in SEQ ID NO: 2 or a protease having at least 85%, at least 90%, at least 95%, at least 96%, at least 97%, at least 98%, or at least 99%, or 100% sequence identity to the polypeptide of SEQ ID NO: 2.

In another embodiment the protease is selected from a Thermococcos sp. PK 58A protease shown in SEQ ID NO: 3 or a protease having at least 85%, at least 90%, at least 95%, at least 96%, at least 97%, at least 98%, or at least 99%, or 100% sequence identity to the polypeptide of SEQ ID NO: 3.

In another embodiment the protease is selected from a variant Thermoascus aurantiacus protease, wherein the variant protease comprises one of the following combinations of mutations:

### D79L+S87P+A112P+D142L;

### D79L+S87P+D142L; or

A27K+D79L+Y82F+S87G+D104P+A112P+A126V+D142L; and the protease variant has at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 4.

The compositions may be prepared in accordance with methods known in the art and may be in the form of a liquid or a dry composition. The compositions may be stabilized in accordance with methods known in the art.

### Uses

Processes for Producing Fermentation Products from Gelatinized Starch-Containing Material

In this aspect the present invention relates to a process for producing a fermentation product, especially ethanol, from starch-containing material, which process includes a liquefaction step and sequentially or simultaneously performed saccharification and fermentation steps.

The invention relates to processes for producing fermentation products from starch-containing material comprising the steps of:

i) liquefying starch-containing material using variant alpha-amylase according to the invention;

ii) saccharifying the liquefied material using a glucoamylase;

iii) fermenting using a fermenting organism.

Protease Present and/or Added During Liquefaction

According to the invention a thermostable protease may in one embodiment be present and/or added during liquefaction together with an alpha-amylase, such as a thermostable alpha-amylase, and optionally a carbohydrate-source generating enzyme, in particular a thermostable glucoamylase or thermostable pullulanase.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

In a preferred embodiment the thermostable protease used according to the invention is a “metallo protease” defined as a protease belonging to EC 3.4.24 (metalloendopeptidases); preferably EC 3.4.24.39 (acid metallo proteinases).

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein). Two protease assays are described below in the “Materials & Methods”-section, of which the so-called “AZCL-Casein Assay” is the preferred assay.

There are no limitations on the origin of the protease used in a process of the invention as long as it fulfills the thermostability properties defined below.

The protease may be a variant of, e.g., a wild-type protease as long as the protease has the thermostability properties defined herein.

In an embodiment the protease has a themostability above 60%, such as above 90%, such as above 100%, such as above 110% at 85° C. as determined using the Zein-BCA assay.

In an embodiment protease has a themostability between 60-120, such as between 70-120%, such as between 80-120%, such as between 90-120%, such as between 100-120%, such as 110-120% at 85° C. as determined using the Zein-BCA assay.

In one embodiment the thermostable protease is a variant of a metallo protease as defined above. In an embodiment the thermostable protease used in a process of the invention is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In an embodiment the thermostable protease is a variant of the mature part of the metallo protease shown in SEQ ID NO: 2 disclosed in WO 2003/048353 or the mature part of SEQ ID NO: 1 in WO 2010/008841 and shown as SEQ ID NO: 4 herein further with mutations selected from below list:

S5*+D79L+S87P+A112P+D142L;

D79L+S87P+A112P+T124V+D142L;

S5*+N26R+D79L+S87P+A112P+D142L;

N26R+T46R+D79L+S87P+A112P+D142L;

T46R+D79L+S87P+T116V+D142L;

D79L+P81R+S87P+A112P+D142L;

A27K+D79L+S87P+A112P+T124V+D142L;

D79L+Y82F+S87P+A112P+T124V+D142L;

D79L+Y82F+S87P+A112P+T124V+D142L;

D79L+S87P+A112P+T124V+A126V+D142L;

D79L+S87P+A112P+D142L;

D79L+Y82F+S87P+A112P+D142L;

S38T+D79L+S87P+A112P+A126V+D142L;

D79L+Y82F+S87P+A112P+A126V+D142L;

A27K+D79L+S87P+A112P+A126V+D142L;

D79L+S87P+N98C+A112P+G135C+D142L;

D79L+S87P+A112P+D142L+T141C+M161C;

S36P+D79L+S87P+A112P+D142L;

A37P+D79L+S87P+A112P+D142L;

S49P+D79L+S87P+A112P+D142L;

S50P+D79L+S87P+A112P+D142L;

D79L+S87P+D104P+A112P+D142L;

D79L+Y82F+S87G+A112P+D142L;

S70V+D79L+Y82F+S87G+Y97W+A112P+D142L;

D79L+Y82F+S87G+Y97W+D104P+A112P+D142L;

S70V+D79L+Y82F+S87G+A112P+D142L;

D79L+Y82F+S87G+D104P+A112P+D142L;

D79L+Y82F+S87G+A112P+A126V+D142L;

Y82F+S87G+S70V+D79L+D104P+A112P+D142L;

Y82F+S87G+D79L+D104P+A112P+A126V+D142L;

A27K+D79L+Y82F+S87G+D104P+A112P+A126V+D142L;

A27K+Y82F+S87G+D104P+A112P+A126V+D142L;

A27K+D79L+Y82F+D104P+A112P+A126V+D142L;

A27K+Y82F+D104P+A112P+A126V+D142L;

A27K+D79L+S87P+A112P+D142L;

D79L+S87P+D142L.

In a preferred embodiment the thermostable protease is a variant of the metallo protease disclosed as the mature part of SEQ ID NO: 2 disclosed in WO 2003/048353 or the mature part of SEQ ID NO: 1 in WO 2010/008841 or SEQ ID NO: 4 herein with the following mutations:

### D79L+S87P+A112P+D142L;

### D79L+S87P+D142L; or

### A27K+D79L+Y82F+S87G+D104P+A112P+A126V+D142L.

In an embodiment the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841 or SEQ ID NO: 4 herein.

The thermostable protease may also be derived from any bacterium as long as the protease has the thermostability properties defined below.

In an embodiment the thermostable protease is derived from a strain of the bacterium Pyrococcus, such as a strain of Pyrococcus furiosus (pfu protease).

In an embodiment the protease is one shown as SEQ ID NO: 1 in U.S. Pat. No. 6,358,726 (Takara Shuzo Company) and SEQ ID NO: 2 herein.

In another embodiment the thermostable protease is one disclosed in SEQ ID NO: 2 herein or a protease having at least 80% identity, such as at least 85%, such as at least 90%, such as at least 95%, such as at least 96%, such as at least 97%, such as at least 98%, such as at least 99% identity to SEQ ID NO: 1 in U.S. Pat. No. 6,358,726 or SEQ ID NO: 2 herein. The Pyroccus furiosus protease can be purchased from Takara Bio, Japan.

In another embodiment the enzyme composition of the invention, comprises a Thermococcus sp. PK 58A protease having at least 80%, such as at least 85%, such as at least 90%, such as at least 95%, such as at least 96%, such as at least 97%, such as at least 98%, such as at least 99%, or at least 100% identity to amino acids 107 to 425 or amino acids 108-425 or amino acids 109-425 of SEQ ID NO: 3.

Glucoamylase Present and/or Added in Liquefaction

In an embodiment a glucoamylase is present and/or added in liquefaction step a) in a process of the invention (i.e., oil recovery process and fermentation product production process).

In a preferred embodiment the glucoamylase present and/or added in liquefaction step a) is derived from a strain of the genus Penicillium, especially a strain of Penicillium oxalicum disclosed as SEQ ID NO: 2 in WO 2011/127802 or SEQ ID NO: 5 herein.

In an embodiment the glucoamylase has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% or 100% identity to the mature polypeptide shown in SEQ ID NO: 2 in WO 2011/127802 or SEQ ID NO: 5 herein.

In a preferred embodiment the glucoamylase is a variant of the Penicillium oxalicum glucoamylase shown in SEQ ID NO: 2 in WO 2011/127802 or SEQ ID NO: 5 herein having a K79V substitution (using the mature sequence shown in SEQ ID NO: 5 for numbering), such as a variant disclosed in WO 2013/053801.

In an embodiment the Penicillium oxalicum glucoamylase has a K79V substitution (using SEQ ID NO: 5 for numbering) and preferably further one of the following substitutions:

### T65A; or

### Q327F; or

### E501V; or

### Y504T; or

### Y504*; or

### T65A+Q327F; or

### T65A+E501V; or

### T65A+Y504T; or

### T65A+Y504*; or

### Q327F+E501V; or

### Q327F+Y504T; or

### Q327F+Y504*; or

### E501V+Y504T; or

### E501V+Y504*; or

### T65A+Q327F+E501V; or

### T65A+Q327F+Y504T; or

### T65A+E501V+Y504T; or

### Q327F+E501V+Y504T; or

### T65A+Q327F+Y504*; or

### T65A+E501V+Y504*; or

### Q327F+E501V+Y504*; or

### T65A+Q327F+E501V+Y504T; or

### T65A+Q327F+E501V+Y504*;

### E501V+Y504T; or

### T65A+K161S; or

### T65A+Q405T; or

### T65A+Q327W; or

### T65A+Q327F; or

### T65A+Q327Y; or

### P11F+T65A+Q327F; or

### R1K+D3W+K5Q+G7V+N8S+T10K+P11S+T65A+Q327F; or

### P2N+P4S+P11F+T65A+Q327F; or

### P11F+D26C+K330+T65A+Q327F; or

### P2N+P4S+P11F+T65A+Q327W+E501V+Y504T; or

### R1E+D3N+P4G+G6R+G7A+N8A+T10D+P11D+T65A+Q327F; or

### P11F+T65A+Q327W; or

### P2N+P4S+P11F+T65A+Q327F+E501V+Y504T; or

### P11F+T65A+Q327W+E501V+Y504T; or

### T65A+Q327F+E501V+Y504T; or

### T65A+S105P+Q327W; or

### T65A+S105P+Q327F; or

### T65A+Q327W+S364P; or

### T65A+Q327F+S364P; or

### T65A+S103N+Q327F; or

### P2N+P4S+P11F+K34Y+T65A+Q327F; or

### P2N+P4S+P11F+T65A+Q327F+D445N+V447S; or

### P2N+P4S+P11F+T65A+I172V+Q327F; or

### P2N+P4S+P11F+T65A+Q327F+N502*; or

### P2N+P4S+P11F+T65A+Q327F+N502T+P563S+K571E; or

### P2N+P4S+P11F+R31S+K33V+T65A+Q327F+N564D+K571S; or

### P2N+P4S+P11F+T65A+Q327F+S377T; or

### P2N+P4S+P11F+T65A+V325T+Q327W; or

### P2N+P4S+P11F+T65A+Q327F+D445N+V447S+E501V+Y504T; or

### P2N+P4S+P11F+T65A+1172V+Q327F+E501V+Y504T; or

### P2N+P4S+P11F+T65A+Q327F+S377T+E501V+Y504T; or

### P2N+P4S+P11F+D26N+K34Y+T65A+Q327F; or

### P2N+P4S+P11F+T65A+Q327F+I375A+E501V+Y504T; or

### P2N+P4S+P11F+T65A+K218A+K221D+Q327F+E501V+Y504T; or

### P2N+P4S+P11F+T65A+S103N+Q327F+E501V+Y504T; or

### P2N+P4S+T10D+T65A+Q327F+E501V+Y504T; or

### P2N+P4S+F12Y+T65A+Q327F+E501V+Y504T; or

### K5A+P11F+T65A+Q327F+E501V+Y504T; or

### P2N+P4S+T10E+E18N+T65A+Q327F+E501V+Y504T; or

### P2N+T10E+E18N+T65A+Q327F+E501V+Y504T; or

### P2N+P4S+P11F+T65A+Q327F+E501V+Y504T+T568N; or

### P2N+P4S+P11F+T65A+Q327F+E501V+Y504T+K524T+G526A; or

### P2N+P4S+P11F+K34Y+T65A+Q327F+D445N+V447S+E501V+Y504T; or

### P2N+P4S+P11F+R31S+K33V+T65A+Q327F+D445N+V447S+E501V+Y504T; or

### P2N+P4S+P11F+D26N+K34Y+T65A+Q327F+E501V+Y504T; or

### P2N+P4S+P11F+T65A+F80*+Q327F+E501V+Y504T; or

### P2N+P4S+P11F+T65A+K112S+Q327F+E501V+Y504T; or

### P2N+P4S+P11F+T65A+Q327F+E501V+Y504T+T516P+K524T+G526A; or

### P2N+P4S+P11F+T65A+Q327F+E501V+N502T+Y504*; or

### P2N+P4S+P11F+T65A+Q327F+E501V+Y504T; or

### P2N+P4S+P11F+T65A+S103N+Q327F+E501V+Y504T; or

### K5A+P11F+T65A+Q327F+E501V+Y504T; or

### P2N+P4S+P11F+T65A+Q327F+E501V+Y504T+T516P+K524T+G526A; or

### P2N+P4S+P11F+T65A+K79A+Q327F+E501V+Y504T; or

### P2N+P4S+P11F+T65A+K79G+Q327F+E501V+Y504T; or

### P2N+P4S+P11F+T65A+K791+Q327F+E501V+Y504T; or

### P2N+P4S+P11F+T65A+K79L+Q327F+E501V+Y504T; or

### P2N+P4S+P11F+T65A+K79S+Q327F+E501V+Y504T; or

### P2N+P4S+P11F+T65A+L72V+Q327F+E501V+Y504T; or

### S255N+Q327F+E501V+Y504T; or

### P2N+P4S+P11F+T65A+E74N+V79K+Q327F+E501V+Y504T; or

### P2N+P4S+P11F+T65A+G220N+Q327F+E501V+Y504T; or

### P2N+P4S+P11F+T65A+Y245N+Q327F+E501V+Y504T; or

### P2N+P4S+P11F+T65A+Q253N+Q327F+E501V+Y504T; or

### P2N+P4S+P11F+T65A+D279N+Q327F+E501V+Y504T; or

### P2N+P4S+P11F+T65A+Q327F+S359N+E501V+Y504T; or

### P2N+P4S+P11F+T65A+Q327F+D370N+E501V+Y504T; or

### P2N+P4S+P11F+T65A+Q327F+V460S+E501V+Y504T; or

### P2N+P4S+P11F+T65A+Q327F+V460T+P468T+E501V+Y504T; or

### P2N+P4S+P11F+T65A+Q327F+T463N+E501V+Y504T; or

### P2N+P4S+P11F+T65A+Q327F+S465N+E501V+Y504T; or

### P2N+P4S+P11F+T65A+Q327F+T477N+E501V+Y504T.

In a preferred embodiment the glucoamylase present and/or added in liquefaction is the Penicillium oxalicum glucoamylase having a K79V substitution and preferably further one of the following substitutions:

P11F+T65A+Q327F;

P2N+P4S+P11F+T65A+Q327F (using SEQ ID NO: 5 for numbering).

In an embodiment the glucoamylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 5 herein.

The glucoamylase may be added in amounts from 0.1-100 micro grams EP/g, such as 0.5-50 micro grams EP/g, such as 1-25 micrograms EP/g, such as 2-12 micrograms EP/g DS.

Glucoamylase Present and/or Added in Saccharification and/or Fermentation

A glucoamylase is present and/or added in saccharification and/or fermentation, preferably simultaneous saccharification and fermentation (SSF), in a process of the invention (i.e., oil recovery process and fermentation product production process).

In an embodiment the glucoamylase present and/or added in saccharification and/or fermentation is of fungal origin, preferably from a stain of Aspergillus, preferably A. niger, A. awamori, or A. oryzae; or a strain of Trichoderma, preferably T. reesei; or a strain of Talaromyces, preferably T. emersonii or a strain of Trametes, preferably T. cingulata, or a strain of Pycnoporus, or a strain of Gloeophyllum, such as G. serpiarium or G. trabeum, or a strain of the Nigrofomes.

In an embodiment the glucoamylase is derived from Talaromyces, such as a strain of Talaromyces emersonii, such as the one shown in SEQ ID NO: 6 herein,

In an embodiment the glucoamylase is selected from the group consisting of:

(i) a glucoamylase comprising the polypeptide of SEQ ID NO: 6 herein;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6 herein.

In an embodiment the glucoamylase is derived from a strain of the genus Pycnoporus, in particular a strain of Pycnoporus sanguineus described in WO 2011/066576 (SEQ ID NOs 2, 4 or 6), such as the one shown as SEQ ID NO: 4 in WO 2011/066576.

In an embodiment the glucoamylase is derived from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16). In a preferred embodiment the glucoamylase is the Gloeophyllum sepiarium shown in SEQ ID NO: 2 in WO 2011/068803 or SEQ ID NO: 7 herein.

In a preferred embodiment the glucoamylase is derived from Gloeophyllum serpiarium, such as the one shown in SEQ ID NO: 7 herein. In an embodiment the glucoamylase is selected from the group consisting of:

(i) a glucoamylase comprising the polypeptide of SEQ ID NO: 7 herein;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 7 herein.

In another embodiment the glucoamylase is derived from Gloeophyllum trabeum such as the one shown in SEQ ID NO: 8 herein. In an embodiment the glucoamylase is selected from the group consisting of:

(i) a glucoamylase comprising the polypeptide of SEQ ID NO: 8 herein;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 8 herein.

In an embodiment the glucoamylase is derived from a strain of the genus Nigrofomes, in particular a strain of Nigrofomes sp. disclosed in WO 2012/064351.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL and AMG™ E (from Novozymes A/S); OPTIDEX™ 300, GC480, GC417 (from DuPont.); AMIGASE™ and AMIGASE™ PLUS (from DSM); G-ZYME™ G900, G-ZYME™ and G990 ZR (from DuPont).

According to a preferred embodiment of the invention the glucoamylase is present and/or added in saccharification and/or fermentation in combination with an alpha-amylase. Examples of suitable alpha-amylase are described below.

Alpha-Amylase Present and/or Added in Saccharification and/or Fermentation

In an embodiment an alpha-amylase is present and/or added in saccharification and/or fermentation in a process of the invention. In a preferred embodiment the alpha-amylase is of fungal or bacterial origin. In a preferred embodiment the alpha-amylase is a fungal acid stable alpha-amylase. A fungal acid stable alpha-amylase is an alpha-amylase that has activity in the pH range of 3.0 to 7.0 and preferably in the pH range from 3.5 to 6.5, including activity at a pH of about 4.0, 4.5, 5.0, 5.5, and 6.0.

In a preferred embodiment the alpha-amylase present and/or added in saccharification and/or fermentation is derived from a strain of the genus Rhizomucor, preferably a strain the Rhizomucor pusillus, such as one shown in SEQ ID NO: 3 in WO 2013/006756, such as a Rhizomucor pusillus alpha-amylase hybrid having an Aspergillus niger linker and starch-binding domain, such as the one shown in SEQ ID NO: 9 herein, or a variant thereof.

In an embodiment the alpha-amylase present and/or added in saccharification and/or fermentation is selected from the group consisting of:

(i) an alpha-amylase comprising the polypeptide of SEQ ID NO: 9 herein;

(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 9 herein.

In a preferred embodiment the alpha-amylase is a variant of the alpha-amylase shown in SEQ ID NO: 9 having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R+V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 9 for numbering).

In an embodiment the alpha-amylase is derived from a Rhizomucor pusillus with an Aspergillus niger glucoamylase linker and starch-binding domain (SBD), preferably disclosed as SEQ ID NO: 9 herein, preferably having one or more of the following substitutions: G128D, D143N, preferably G128D+D143N (using SEQ ID NO: 9 for numbering).

In an embodiment the alpha-amylase variant present and/or added in saccharification and/or fermentation has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 9 herein.

In a preferred embodiment the ratio between glucoamylase and alpha-amylase present and/or added during saccharification and/or fermentation may preferably be in the range from 500:1 to 1:1, such as from 250:1 to 1:1, such as from 100:1 to 1:1, such as from 100:2 to 100:50, such as from 100:3 to 100:70.

Pullulanase Present and/or Added in Liquefaction and/or Saccharification and/or Fermentation.

A pullulanase may be present and/or added during liquefaction step a) and/or saccharification step b) or fermentation step c) or simultaneous saccharification and fermentation.

Pullulanases (E.C. 3.2.1.41, pullulan 6-glucano-hydrolase), are debranching enzymes characterized by their ability to hydrolyze the alpha-1,6-glycosidic bonds in, for example, amylopectin and pullulan.

Contemplated pullulanases according to the present invention include the pullulanases from Bacillus amyloderamificans disclosed in U.S. Pat. No. 4,560,651 (hereby incorporated by reference), the pullulanase disclosed as SEQ ID NO: 2 in WO 01/51620 (hereby incorporated by reference), the Bacillus deramificans disclosed as SEQ ID NO: 4 in WO 01/51620 (hereby incorporated by reference), and the pullulanase from Bacillus acidopullulyticus disclosed as SEQ ID NO: 6 in WO 01/51620 (hereby incorporated by reference) and also described in FEMS Mic. Let. 115: 97-106 (1994).

The pullulanase may according to the invention be added in an effective amount which include the preferred amount of about 0.0001-10 mg enzyme protein per gram DS, preferably 0.0001-0.10 mg enzyme protein per gram DS, more preferably 0.0001-0.010 mg enzyme protein per gram DS. Pullulanase activity may be determined as NPUN. An Assay for determination of NPUN is described in the “Materials & Methods”-section below.

Suitable commercially available pullulanase products include PROMOZYME D, PROMOZYME™ D2 (Novozymes A/S, Denmark), OPTIMAX L-300 (Genencor Int., USA), and AMANO 8 (Amano, Japan).

The fermentation product, such as especially ethanol, may optionally be recovered after fermentation, e.g., by distillation. Suitable starch-containing starting materials are listed in the section “Starch-Containing Materials”-section below. In an embodiment the starch-containing materials is corn or wheat.

The fermenting organism is preferably yeast, preferably a strain of Saccharomyces, especially a strain of Saccharomyces cerevisae. Suitable fermenting organisms are listed in the “Fermenting Organisms”-section above. In a preferred embodiment steps ii) and iii) are carried out sequentially or simultaneously (i.e., as SSF process). The aqueous slurry may contain from 10-55 wt.-% dry solids, preferably 25-45 wt.-% dry solids, more preferably 30-40 wt.-% dry solids of starch-containing material. The slurry is heated to above the initial gelatinization temperature. Alpha-amylase, preferably bacterial alpha-amylase, may be added to the slurry. In an embodiment the slurry is also jet-cooked to further gelatinize the slurry before being subjected to an alpha-amylase in liquefaction step i).

The temperature during step (i) is above the initial gelatinization temperature, such as between 80-90° C., such as around 85° C.

In an embodiment liquefaction is carried out as a three-step hot slurry process. The slurry is heated to between 60-95° C., preferably between 80-90° C., and alpha-amylase is added to initiate liquefaction (thinning). Then the slurry is jet-cooked at a temperature between 95-140° C., preferably 105-125° C., for 1-15 minutes, preferably for 3-10 minutes, especially around 5 minutes. The slurry is cooled to 60-95° C., preferably 80-90° C., and more alpha-amylase is added to finalize hydrolysis (secondary liquefaction). The liquefaction process is usually carried out at pH 4.5-6.5, such as around 4.8, or a pH between 5.0-6.2, such as 5.0-6.0, such as between 5.0-5.5, such as around 5.2, such as around 5.4, such as around 5.6, such as around 5.8. Milled and liquefied starch is known as “mash”.

The saccharification in step ii) may be carried out using conditions well known in the art. For instance, a full saccharification process may last up to from about 24 to about 72 hours. In an embodiment a pre-saccharification step is done at 40-90 minutes at a temperature between 30-65° C., typically at about 60° C., followed by complete saccharification during fermentation in a simultaneous saccharification and fermentation step (SSF). Saccharification is typically carried out at temperatures from 30-70° C., such as 55-65° C., typically around 60° C., and at a pH between 4 and 5, normally at about pH 4.5.

The most widely used process in fermentation product production, especially ethanol production, is simultaneous saccharification and fermentation (SSF) process, in which there is no holding stage for the saccharification.

SSF may typically be carried out at a temperature between 25° C. and 40° C., such as between 28° C. and 36° C., such as between 30° C. and 34° C., such as around 32° C., when the fermentation organism is yeast, such as a strain of Saccharomyces cerevisiae, and the desired fermentation product is ethanol. In an embodiment fermentation is ongoing for 6 to 120 hours, in particular 24 to 96 hours.

Other fermentation products may be fermented at conditions and temperatures, well known to the skilled person in the art, suitable for the fermenting organism in question.

### Fermentation Medium

The environment in which fermentation is carried out is often referred to as the “fermentation media” or “fermentation medium”. The fermentation medium includes the fermentation substrate, that is, the carbohydrate source that is metabolized by the fermenting organism. The fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

### Fermenting Organisms

The term “fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

### Starch-Containing Materials

Any suitable starch-containing material may be used according to the present invention. The starting material is generally selected based on the desired fermentation product. Examples of starch-containing materials, suitable for use in a process of the invention, include whole grains, corn, wheat, barley, rye, milo, sago, cassava, tapioca, sorghum, rice, peas, beans, or sweet potatoes, or mixtures thereof or starches derived therefrom, or cereals. Contemplated are also waxy and non-waxy types of corn and barley. In a preferred embodiment the starch-containing material, used for ethanol production according to the invention, is corn or wheat.

### Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

### Recovery of Fermentation Products

Subsequent to fermentation, or SSF, the fermentation product may be separated from the fermentation medium. The slurry may be distilled to extract the desired fermentation product (e.g., ethanol). Alternatively the desired fermentation product may be extracted from the fermentation medium by micro or membrane filtration techniques. The fermentation product may also be recovered by stripping or other method well known in the art.

The present invention is further defined in the following list of embodiments:

### Embodiment 1

An alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 85%, at least 90%, at least 95%, at least 96%, at least 97%, at least 98%, or at least 99%, but less than 100% sequence identity to the polypeptide of SEQ ID NO: 1, and wherein the variant comprises at least one of the following combination of substitutions:

### V59A+E129V+K177L+R179E+H208Y+K220P+N224L+Q254S;

### V59A+E129V+K177L+R179E+Q254S+M284V; or

V59A+E129V+K177L+R179E+V212T+Q254S+M284V (using SEQ ID NO: 1 for numbering); and wherein the variants have alpha-amylase activity.

### Embodiment 2

The variant of embodiment 1, further having a double deletion of two amino acids in the region from position 180 to 182, e.g., at positions G180, I181 and/or G182, in particular deletion of I181+G182, G180+I181, or G180+G182.

### Embodiment 3

The variant of embodiment 1, wherein the double deletion is I181+G182.

### Embodiment 4

The variant of embodiment 1, wherein the amino acid at position 89 is not R, particularly the amino acid at position 89 is selected from N, D, E, H, S, T, A, or Q, more particularly 89Q.

### Embodiment 5

The variant of any of embodiments 1-4, further comprising a substitution N193F.

### Embodiment 6

The variant of any of embodiments 1-5, wherein when said variant is added in liquefaction at pH 4.8 and a temperature of 85° C. for 2 hours the resulting liquefact has reduced viscosity compared to using a parent alpha-amylase.

### Embodiment 7

The variant of embodiment 6, wherein when said variant is added in liquefaction at pH 4.8 and a temperature of 85° C. for 2 hours the resulting ethanol yield after subsequent saccharification and fermentation is equivalent to using a parent alpha-amylase.

### Embodiment 8

The variant of embodiment 6 and 7, wherein the parent alpha-amylase is identical to the variant except for the presence of a substitution at position 89, in particular Q89R.

### Embodiment 9

The variant of any of embodiments 1-8, wherein the reduction in viscosity is at least 5%, at least at least 6%, at least 7%, at least 8%, at least 9%, or at least 10%, at least 11%, at least 12%, at least 13%, at least 14%, at least 15%, compared to the parent alpha-amylase.

### Embodiment 10

The variant of any of embodiments 1-9, wherein the Bacillus stearothermophilus variant alpha-amylase is truncated, preferably to have a length of around 490 amino acids, such as from 482-493 amino acids.

### Embodiment 11

The variant of any of embodiments 1-9, wherein the Bacillus stearothermophilus variant alpha-amylase is truncated, preferably after position 484 of SEQ ID NO: 1, particularly after position 485, particularly after position 486, particularly after position 487, particularly after position 488, particularly after position 489, particularly after position 490, particularly after position 491, particularly after position 492, more particularly after position 493.

### Embodiment 12

An polynucleotide encoding the variant of any of embodiments 1-11.

### Embodiment 13

A nucleic acid construct comprising the polynucleotide of embodiment 12.

### Embodiment 14

An expression vector comprising the polynucleotide of embodiment 12.

### Embodiment 15

A host cell comprising the polynucleotide of embodiment 12.

### Embodiment 16

A method of producing an alpha-amylase variant of embodiments 1-11, comprising:

a) cultivating the host cell of embodiment 15 under conditions suitable for expression of the variant; and

b) recovering the variant.

### Embodiment 17

A process for producing fermentation products from starch-containing material comprising the steps of:

a) liquefying the starch-containing material at a temperature above the initial gelatinization temperature using an alpha-amylase according to any of embodiments 1-11;

b) saccharifying using a glucoamylase;

c) fermenting using a fermenting organism.

### Embodiment 18

The process according to embodiment 17, wherein step a) further comprises adding a protease.

### Embodiment 19

The process according to embodiment 18, wherein the protease is selected from, a Pyrococcus sp. protease, a Thermococcus sp. S8A protease, or a Thermoascus sp. protease.

### Embodiment 20

The process according to embodiment 18 or 19, wherein the protease is a Pyrococcus furiosus protease S, or a Thermococcos sp PK S8A protease, or a variant Thermoascus aurantiacus protease, especially Thermoascus aurantiacus CGMCC No. 0670.

### Embodiment 21

The process of embodiment 20, wherein a glucoamylase is present and/or added in liquefaction step a).

### Embodiment 22

The process of embodiment 21, wherein the glucoamylase present and/or added in liquefaction step a) is derived from a strain of the genus Penicillium, especially a strain of Penicillium oxalicum disclosed as SEQ ID NO: 5.

### Embodiment 23

The process of embodiment 22, wherein the glucoamylase has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% or 100% identity to the polypeptide of SEQ ID NO: 5.

### Embodiment 24

The process of embodiment 22 or 23, wherein the glucoamylase is a variant of the Penicillium oxalicum glucoamylase shown in SEQ ID NO: 5 further having a K79V substitution (using the sequence shown in SEQ ID NO: 5 for numbering).

### Embodiment 25

The process of any of embodiments 22-24, wherein the glucoamylase present and/or added in liquefaction is the Penicillium oxalicum glucoamylase having a K79V substitution (using SEQ ID NO: 6 for numbering) and further one of the following combinations of substitutions:

P11F+T65A+Q327F

P2N+P4S+P11F+T65A+Q327F (using SEQ ID NO: 5 for numbering).

### Embodiment 26

The process of embodiment 24 or 25, wherein the glucoamylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 5.

### Embodiment 27

The process of any of embodiments 17-26, wherein a pullulanase is present and/or added in liquefaction step a).

### Embodiment 28

The process of any of embodiments 17-27, wherein the glucoamylase present and/or added in saccharification step b) and/or fermentation step c) is of fungal origin, preferably from a stain of Aspergillus, preferably A. niger, A. awamori, or A. oryzae; or a strain of Trichoderma, preferably T. reesei; or a strain of Talaromyces, preferably T. emersonii, or a strain of Trametes, preferably T. cingulata, or a strain of Pycnoporus, or a strain of Gloeophyllum, such as G. sepiarium or G. trabeum, or a strain of the Nigrofomes.

### Embodiment 29

The process of embodiment 28, wherein the glucoamylase is derived from Talaromyces emersonii, such as the one shown in SEQ ID NO: 6.

### Embodiment 30

The process of embodiment 29, wherein the glucoamylase is selected from the group consisting of:

(i) a glucoamylase comprising the polypeptide of SEQ ID NO: 6;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 6.

### Embodiment 31

The process of embodiment 28, wherein the glucoamylase is derived from Gloeophyllum sepiarium, such as the one shown in SEQ ID NO: 7.

### Embodiment 32

The process of embodiment 31, wherein the glucoamylase is selected from the group consisting of:

(i) a glucoamylase comprising the polypeptide of SEQ ID NO: 7;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 7.

### Embodiment 33

The process of embodiment 28, wherein the glucoamylase is derived from Gloeophyllum trabeum such as the one shown in SEQ ID NO: 8.

### Embodiment 34

The process of embodiment 33, wherein the glucoamylase is selected from the group consisting of:

(i) a glucoamylase comprising the polypeptide of SEQ ID NO: 8;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 8.

### Embodiment 35

The process of any of embodiments 17-34, wherein the glucoamylase is present in saccharification and/or fermentation in combination with an alpha-amylase.

### Embodiment 36

The process of embodiment 35, wherein the alpha-amylase present in saccharification and/or fermentation is of fungal or bacterial origin.

### Embodiment 37

The process of embodiment 35 or 36, wherein the alpha-amylase present and/or added in saccharification and/or fermentation is derived from a strain of the genus Rhizomucor, preferably a strain of Rhizomucor pusillus, such as a Rhizomucor pusillus alpha-amylase hybrid having an Aspergillus niger linker and starch-bonding domain, such as the one shown in SEQ ID NO: 9.

### Embodiment 38

The process of embodiment 37, wherein the alpha-amylase present in saccharification and/or fermentation is selected from the group consisting of:

(i) an alpha-amylase comprising the polypeptide of SEQ ID NO: 9;

(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 9.

### Embodiment 39

The process of any of embodiments 35-38, wherein the alpha-amylase is derived from a Rhizomucor pusillus with an Aspergillus niger glucoamylase linker and starch-binding domain (SBD), preferably disclosed as SEQ ID NO: 9, preferably having one or more of the following substitutions: G128D, D143N, preferably G128D+D143N.

### Embodiment 40

The process of embodiment 39, wherein the alpha-amylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 9.

### Embodiment 41

The process of any of embodiments 17-40, further comprising, prior to the liquefaction step a), the steps of:

i) reducing the particle size of the starch-containing material, preferably by dry milling;

ii) forming a slurry comprising the starch-containing material and water.

### Embodiment 42

The process of any of embodiments 17-41, wherein the pH in liquefaction is between above 4.5-6.5, such as around 4.8, or a pH between 5.0-6.2, such as 5.0-6.0, such as between 5.0-5.5, such as around 5.2, such as around 5.4, such as around 5.6, such as around 5.8.

### Embodiment 43

The process of any of embodiments 17-42, wherein the temperature in liquefaction is above the initial gelatinization temperature, such as in the range from 70-100° C., such as between 75-95° C., such as between 75-90° C., preferably between 80-90° C., especially around 85° C.

### Embodiment 44

The process of any of embodiments 17-43, wherein the fermentation product is an alcohol, preferably ethanol, especially fuel ethanol, potable ethanol and/or industrial ethanol.

### Embodiment 45

The process of any of embodiments 17-44, wherein the starch-containing material is derived from corn, wheat, barley, rye, milo, sago, cassava, manioc, tapioca, sorghum, rice or potatoes.

### Embodiment 46

An enzyme composition comprising the variant alpha-amylase of any of embodiments 1-11.

### Embodiment 47

The enzyme composition of embodiment 46, further comprising a protease.

### Embodiment 48

The composition of embodiment 47, wherein the protease is selected from a Pyrococcus furiosus protease S shown in SEQ ID NO: 2 or a protease having at least 85%, at least 90%, at least 95%, at least 96%, at least 97%, at least 98%, or at least 99%, or 100% sequence identity to the polypeptide of SEQ ID NO: 2.

### Embodiment 49

The composition of embodiment 47, wherein the protease is selected from a Thermococcos sp. PK S8A protease shown in SEQ ID NO: 3 or a protease having at least 85%, at least 90%, at least 95%, at least 96%, at least 97%, at least 98%, or at least 99%, or 100% sequence identity to the polypeptide of SEQ ID NO: 3.

### Embodiment 50

The composition of embodiment 47, wherein the protease is selected from a variant Thermoascus aurantiacus protease, wherein the variant protease comprises one of the following combinations of mutations:

### D79L+S87P+A112P+D142L;

### D79L+S87P+D142L; or

A27K+D79L+Y82F+S87G+D104P+A112P+A126V+D142L; and the protease variant has at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 4.

The present invention is further described by the following examples that should not be construed as limiting the scope of the invention.

### EXAMPLES

### Example 1—Assays for Alpha-Amylase Activity pNP-G7 Assay

The alpha-amylase activity may be determined by a method employing the G7-pNP substrate. G7-pNP which is an abbreviation for 4,6-ethylidene(G₇)-p-nitrophenyl(G₁)-α,D-maltoheptaoside, a blocked oligosaccharide which can be cleaved by an endo-amylase, such as an alpha-amylase. Following the cleavage, the alpha-Glucosidase included in the kit digest the hydrolysed substrate further to liberate a free PNP molecule which has a yellow color and thus can be measured by visible spectophometry at λ=405 nm (400-420 nm). Kits containing G7-pNP substrate and alpha-Glucosidase is manufactured by Roche/Hitachi (cat. no. 11876473).

### Reagents:

The G7-pNP substrate from this kit contains 22 mM 4,6-ethylidene-G7-pNP and 52.4 mM HEPES (2-[4-(2-hydroxyethyl)-1-piperazinyl]-ethanesulfonic acid), pH 7.0).

The alpha-Glucosidase reagent contains 52.4 mM HEPES, 87 mM NaCl, 12.6 mM MgCl₂, 0.075 mM CaCl₂, ≥4 kU/L alpha-glucosidase).

The substrate working solution is made by mixing 1 mL of the alpha-Glucosidase reagent with 0.2 mL of the G7-pNP substrate. This substrate working solution is made immediately before use.

Dilution buffer: 50 mM MOPS, 0.05% (w/v) Triton X100 (polyethylene glycol p-(1,1,3,3-tetramethylbutyl)-phenyl ether (C₁₄H₂₂O(C₂H₄O)ₙ (n=9-10))), 1 mM CaCl₂, pH8.0.

### Procedure:

The amylase sample to be analyzed is diluted in dilution buffer to ensure the pH in the diluted sample is 7. The assay is performed by transferring 20 μl diluted enzyme samples to 96 well microtiter plate and adding 80 μl substrate working solution. The solution is mixed and pre-incubated 1 minute at room temperature and absorption is measured every 20 seconds over 5 minutes at OD 405 nm.

The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

### Phadebas Activity Assay:

The alpha-amylase activity may also be determined by a method using the Phadebas substrate (from for example Magle Life Sciences, Lund, Sweden). A Phadebas tablet includes interlinked starch polymers that are in the form of globular microspheres that are insoluble in water. A blue dye is covantly bound to these microspheres. The interlinked starch polymers in the microsphere are degraded at a speed that is proportional to the alpha-amylase activity. When the alpha-amylase degrades the starch polymers, the released blue dye is water soluble and concentration of dye can be determined by measuring absorbance at 620 nm. The concentration of blue is proportional to the alpha-amylase activity in the sample.

The amylase sample to be analysed is diluted in activity buffer with the desired pH. One substrate tablet is suspended in 5 mL activity buffer and mixed on magnetic stirrer. During mixing of substrate transfer 150 μl to microtiter plate (MTP) or PCR-MTP. Add 30 μl diluted amylase sample to 150 μl substrate and mix. Incubate for 15 minutes at 37° C. The reaction is stopped by adding 30 μl 1 M NaOH and mix. Centrifuge MTP for 5 minutes at 4000×g. Transfer 100 μl to new MTP and measure absorbance at 620 nm.

The amylase sample should be diluted so that the absorbance at 620 nm is between 0 and 2.2, and is within the linear range of the activity assay.

### Reducing Sugar Activity Assay:

The alpha-amylase activity may also be determined by reducing sugar assay with for example corn starch substrate. The number of reducing ends formed by the alpha-amylase hydrolysing the alpha-1,4-glycosidic linkages in starch is determined by reaction with p-Hydroxybenzoic acid hydrazide (PHBAH). After reaction with PHBAH the number of reducing ends can be measured by absorbance at 405 nm and the concentration of reducing ends is proportional to the alpha-amylase activity in the sample.

The corns starch substrate (3 mg/ml) is solubilised by cooking for 5 minutes in milliQ water and cooled down before assay. For the stop solution prepare a Ka-Na-tartrate/NaOH solution (K—Na-tartrate (Merck 8087) 50 g/l, NaOH 20 g/l) and prepare freshly the stop solution by adding p-Hydroxybenzoic acid hydrazide (PHBAH, Sigma H9882) to Ka-Na-tartrate/NaOH solution to 15 mg/ml.

In PCR-MTP 50 μl activity buffer is mixed with 50 μl substrate. Add 50 μl diluted enzyme and mix. Incubate at the desired temperature in PCR machine for 5 minutes. Reaction is stopped by adding 75 μl stop solution (Ka-Na-tartrate/NaOH/PHBAH). Incubate in PCR machine for 10 minutes at 95° C. Transfer 150 μl to new MTP and measure absorbance at 405 nm.

The amylase sample should be diluted so that the absorbance at 405 nm is between 0 and 2.2, and is within the linear range of the activity assay.

### Enzchek® Assay:

For the determination of residual amylase activity an EnzChek® Ultra Amylase Assay Kit (E33651, Invitrogen, La Jolla, Calif., USA) may be used.

The substrate is a corn starch derivative, DQ™ starch, which is corn starch labeled with BODIPY® FL dye to such a degree that fluorescence is quenched. One vial containing approx. 1 mg lyophilized substrate is dissolved in 100 microliters of 50 mM sodium acetate (pH 4.0). The vial is vortexed for 20 seconds and left at room temperature, in the dark, with occasional mixing until dissolved. Then 900 microliters of 100 mM acetate, 0.01% (w/v) TRITON® X100, 0.125 mM CaCl₂, pH 5.5 is added, vortexed thoroughly and stored at room temperature, in the dark until ready to use. The stock substrate working solution is prepared by diluting 10-fold in residual activity buffer (100 mM acetate, 0.01% (w/v) TRITON® X100, 0.125 mM CaCl₂, pH 5.5). Immediately after incubation the enzyme is diluted to a concentration of 10-20 ng enzyme protein/ml in 100 mM acetate, 0.01% (W/v) TRITON® X100, 0.125 mM CaCl₂, pH 5.5.

For the assay, 25 microliters of the substrate working solution is mixed for 10 second with 25 microliters of the diluted enzyme in a black 384 well microtiter plate. The fluorescence intensity is measured (excitation: 485 nm, emission: 555 nm) once every minute for 15 minutes in each well at 25° C. and the Vₘₐₓ is calculated as the slope of the plot of fluorescence intensity against time. The plot should be linear and the residual activity assay has been adjusted so that the diluted reference enzyme solution is within the linear range of the activity assay.

### Example 2—Assays for Glucoamylase Activity

### Glucoamylase Units, AGU

The Glucoamylase Unit (AGU) is defined as the amount of enzyme, which hydrolyses 1 micromole maltose per minute under the standard conditions (37° C., pH 4.3, substrate: maltose 100 mM, buffer: acetate 0.1 M, reaction time 6 minutes as set out in the glucoamylase incubation below), thereby generating glucose.

| glucoamylase incubation: | |
|---|---|
| Substrate: | maltose 100 mM |
| Buffer: | acetate 0.1M |
| pH: | 4.30 ± 0.05 |
| Incubation temperature: | 37° C. ± 1 |
| Reaction time: | 6 minutes |
| Enzyme working range: | 0.5-4.0 AGU/mL |

The analysis principle is described by 3 reaction steps:

Step 1 is an enzyme reaction:

Glucoamylase (AMG), EC 3.2.1.3 (exo-alpha-1,4-glucan-glucohydrolase), hydrolyzes maltose to form alpha-D-glucose. After incubation, the reaction is stopped with NaOH.

Steps 2 and 3 result in an endpoint reaction:

Glucose is phosphorylated by ATP, in a reaction catalyzed by hexokinase. The glucose-6-phosphate formed is oxidized to 6-phosphogluconate by glucose-6-phosphate dehydrogenase. In this same reaction, an equimolar amount of NAD+ is reduced to NADH with a resulting increase in absorbance at 340 nm. An autoanalyzer system such as Konelab 30 Analyzer (Thermo Fisher Scientific) may be used.

Color reaction

| Tris | approx. 35 | mM |
|---|---|---|
| ATP | 0.7 | mM |
| NAD⁺ | 0.7 | mM |
| Mg²⁺ | 1.8 | mM |
| Hexokinase | >850 | U/L |
| Glucose-6-P-DH | >850 | U/L |
| pH | approx. 7.8 | |
| Temperature | 37.0° C. ± 1.0° C. | |
| Reaction time | 420 | sec |
| Wavelength | 340 | nm |

### Example 3—Liquefaction Viscosity Using (Alpha-Amylase 1489, 1490, 1492)

The purpose of this experiment was to measure the viscosity reducing performance of Alpha-Amylase 1489, 1490, 1492 according to the invention compared to Alpha-Amylase 369 disclosed in WO 2013/082486, measured after liquefaction at pH 4.8 and 5.3, at 85° C. for 2 hours. AA-369 is identical to AA-1490 and 1492 except for the Q89R substitution present in AA-369 and the V212T substitution present in 1492.

### Liquefaction (Labomat)

Each liquefaction received ground corn (86.79% DS), backset (5.69% DS), and tap water targeting a total weight of 120 g at 32.50% Dry Solids (DS). Backset was blended at 30% w/w of total slurry weight. Initial slurry pH was approximately 5.2 and was adjusted to pH 4.8, 5.3 or 5.8 with 10% w/w sodium hydroxide or 40% v/v sulfuric acid prior to liquefaction. All enzymes were added according to the experimental design listed in Table 1 below. Liquefaction took place in a Labomat using the following conditions: 5° C./min ramp, 17 minute ramp, 103 minute hold time, 40 rpm for the entire run, 200 mL stainless steel canisters. After liquefaction, all canisters were cooled in an ice bath to room temperature and prepared for fermentation based on the protocol listed below under SSF.

### Viscosity Measurement (Rapid Visco Analyzer)

A 40 g sample of each mash was transferred to a Super4 Rapid Visco Analyzer (RVA) aluminum sample cup. In the RVA, the samples were heated to 32° C. and stirred at 160 rpm for 6 minutes and 30 seconds. The average viscosity for the last 30 seconds of the method is reported as mash final viscosity in centipoise (cP).

TABLE 1
Liquefaction Experiment Design

| | pH | Amylase | Dose | |
|---|---|---|---|---|
| 1 | 4.8 | AA-369 | 2.1 | μg/g Dry Solids |
| 2 | 5.3 | | | |
| 3 | 4.8 | AA-1489 | 2.1 | μg/g Dry Solids |
| 4 | 5.3 | | | |
| 5 | 4.8 | AA-1490 | 2.1 | μg/g Dry Solids |
| 6 | 5.3 | | | |
| 7 | 4.8 | AA-1492 | 2.1 | μg/g Dry Solids |
| 8 | 5.3 | | | |

### Results:

| Enzymes | pH | Final Viscosity (cP) |
|---|---|---|
| AA-369 | 4.8 | 1332 |
| AA-369 | 5.3 | 1026 |
| AA-1489 | 4.8 | 1145 |
| AA-1489 | 5.3 | 1008 |
| AA-1490 | 4.8 | 1191 |
| AA-1490 | 5.3 | 1011 |
| AA-1492 | 4.8 | 1177 |
| AA-1492 | 5.3 | 1084 |

These results demonstrate that the thermostable alpha-amylase resulted in superior viscosity breakdown than a control Alpha-Amylase (AA-369) at pH 4.8.

### Example 4—EtOH Production Using (Alpha-Amylase 1489, 1490, 1492) for Liquefaction

The purpose of this experiment was to evaluate the application performance of Alpha-Amylase 1489, 1490, 1492 compared to Alpha-Amylase AA-369, added during liquefaction at pH 4.8 and 5.3, at 85° C. for 2 hours.

### Liquefaction (Labomat)

Each liquefaction received ground corn (86.79% DS), backset (5.69% DS), and tap water targeting a total weight of 120 g at 32.50% Dry Solids (DS). Backset was blended at 30% w/w of total slurry weight. Initial slurry pH was approximately 5.2 and was adjusted to pH 4.8 or 5.3 with 10% w/w sodium hydroxide or 40% v/v sulfuric acid prior to liquefaction. All enzymes were added according to the experimental design listed in Table 19 below. Liquefaction took place in a Labomat using the following conditions: 5° C./min ramp, 17 minute ramp, 103 minute hold time, 40 rpm for the entire run, 200 mL stainless steel canisters. After liquefaction, all canisters were cooled in an ice bath to room temperature and prepared for fermentation based on the protocol listed below under SSF.

### Simultaneous Saccharification and Fermentation (SSF)

Each mash was adjusted to pH 5.0 with 10% w/w sodium hydroxide or 40% v/v sulfuric acid. Penicillin was applied to each mash to a total concentration of 3 ppm. Urea was applied to each mash to a total concentration of 700 ppm. SSF tubes were prepared by aliquoting approximately 4.5 g of mash per 15 mL test tubes, pre-drilled to allow CO₂ release.

Glucoamylase blend A (a blend of glucoamylases from Talaromyces emersonii, WO 99/28448, and Trametes cingulata, WO 2006/069289, and an acid fungal hybrid alpha-amylase from Rhizomucor pusillus, WO 2004/055178 and WO 2006/069290) was added at the dose of 0.60 AGU/g Dry Solid, minimal water was added to each tube to normalize solids, and each mash sample received 100 μL of rehydrated yeast. Rehydrated yeast was prepared by mixing 5.5 g of Fermentis RED STAR into 100 mL of 32° C. tap water for at least 15 minutes.

The tubes of mash were incubated in a waterbath at 32° C. for 54 hours and mixed with a vortex at least once every 24 hours.

### HPLC Analysis

Fermentation sampling took place after approximately 54 hours of fermentation. Each sample was deactivated with 50 μL of 40% v/v H₂SO₄, vortexing, centrifuging at 1460×g for 10 minutes, and filtering through a 0.45 μm Whatman PP filter. 54 hour samples were analyzed under HPLC without further dilution. Samples were stored at 4° C. prior to and during HPLC analysis.

| | |
|---|---|
| HPLC | Agilent's 1100/1200 series with Chem station software |
| system | Degasser, Quaternary Pump, Auto-Sampler, Column |
| | Compartment /w Heater |
| | Refractive Index Detector (RI) |
| Column | Bio-Rad HPX-87H Ion Exclusion Column 300 mm × |
| | 7.8 mm part# 125-0140 |
| | Bio-Rad guard cartridge cation H part# 125-0129, Holder |
| | part# 125-0131 |
| Method | 0.005M H₂SO₄ mobile phase |
| | Flow rate: 0.6 ml/min |
| | Column temperature: 65° C. |
| | RI detector temperature: 55° C. |

The method quantified analyte(s) using calibration standards for ethanol (% w/v). A four point calibration including the origin is used for quantification.

Where applicable, data were analyzed using JMP software (Cary, N.C.) with Oneway ANOVA of pairs using Tukey-Kramer HSD or Dunnett's. Error bars denoting the 95% confidence level were established using the calculated 95% confidence interval in JMP.

TABLE 2
Liquefaction Experiment Design

| | pH | Amylase | Dose | |
|---|---|---|---|---|
| 1 | 4.8 | AA-369 | 2.1 | μg/g Dry Solids |
| 2 | 5.3 | | | |
| 3 | 4.8 | AA-1489 | 2.1 | μg/g Dry Solids |
| 4 | 5.3 | | | |
| 5 | 4.8 | AA-1490 | 2.1 | μg/g Dry Solids |
| 6 | 5.3 | | | |
| 7 | 4.8 | AA-1492 | 2.1 | μg/g Dry Solids |
| 8 | 5.3 | | | |

### Results:

### Glucoamylase Blend A

| Enzymes | pH | EtOH g/L | Level |
|---|---|---|---|
| AA-369 | 4.8 | 130.7 | A, B |
| AA-369 | 5.3 | 130.8 | A, B |
| AA-1489 | 4.8 | 130.9 | A, B |
| AA-1489 | 5.3 | 130.9 | A, B |
| AA-1490 | 4.8 | 131.1 | A |
| AA-1490 | 5.3 | 130.8 | A, B |
| AA-1492 | 4.8 | 130.9 | A, B |
| AA-1492 | 5.3 | 130.3 | B |

These results demonstrate that the thermostable alpha-amylase ethanol yields were comparable to control Alpha-Amylase (AA-369).

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. An alpha-amylase variant, comprising a substitution at one or more positions corresponding to positions 59, 129, 177, 179, 208, 212, 220, 224, 254, and 284 of the polypeptide of SEQ ID NO: 1, wherein the variant has at least at least 85%, at least 90%, at least 95%, at least 96%, at least 97%, at least 98%, or at least 99%, but less than 100% sequence identity to the polypeptide of SEQ ID NO: 1, and wherein the variant comprises at least one of the following combination of substitutions:
V59A+E129V+K177L+R179E+H208Y+K220P+N224L+Q254S;
V59A+E129V+K177L+R179E+Q254S+M284V; or
V59A+E129V+K177L+R179E+V212T+Q254S+M284V (using SEQ ID NO: 1 for numbering); and wherein the variants have alpha-amylase activity.

2. The variant of claim 1, further having a double deletion of two amino acids in the region from position 180 to 182.

3. The variant of claim 1, wherein the double deletion is I181+G182.

4. The variant of claim 1, wherein the amino acid at position 89 is not R.

5. The variant of claim 1, further comprising a substitution N193F.

6. The variant of claim 1, wherein when said variant is added in liquefaction at pH 4.8 and a temperature of 85° C. for 2 hours the resulting liquefact has reduced viscosity compared to using a parent alpha-amylase.

7. The variant of claim 6, wherein when said variant is added in liquefaction at pH 4.8 and a temperature of 85° C. for 2 hours the resulting ethanol yield after subsequent saccharification and fermentation is equivalent to using a parent alpha-amylase.

8. The variant of claim 6, wherein the parent alpha-amylase is identical to the variant except for the presence of a substitution at position 89.

9. The variant of claim 1, wherein the reduction in viscosity is at least 5%, at least at least 6%, at least 7%, at least 8%, at least 9%, or at least 10%, at least 11%, at least 12%, at least 13%, at least 14%, at least 15%, compared to the parent alpha-amylase.

10. The variant of claim 1, wherein the Bacillus stearothermophilus variant alpha-amylase is truncated, preferably to have a length of around 490 amino acids, such as from 482-493 amino acids.

11. The variant of claim 1, wherein the Bacillus stearothermophilus variant alpha-amylase is truncated.

12. An polynucleotide encoding the variant of claim 1.

13. A nucleic acid construct comprising the polynucleotide of claim 12.

14. An expression vector comprising the polynucleotide of claim 12.

15. A host cell comprising the polynucleotide of claim 12.

16. A method of producing an alpha-amylase variant of claim 1, comprising:
a. cultivating the host cell of claim 15 under conditions suitable for expression of the variant; and
b. recovering the variant.

17. A process for producing fermentation products from starch-containing material comprising the steps of:
a) liquefying the starch-containing material at a temperature above the initial gelatinization temperature using:
an alpha-amylase according to claim 1;
b) saccharifying using a glucoamylase;
c) fermenting using a fermenting organism.

18. The process according to claim 17, wherein step a) further comprises adding a protease.

19. An enzyme composition comprising the variant alpha-amylase of claim 1.

20. The enzyme composition of claim 19, further comprising a protease.
